(19) United States
(12) Patent Application Publication
(10) Pub. No.: US 2010/0176212 A1
(43) Pub. Date: Jul. 15, 2010

(54) Central scent distribution system

(76) Inventors: Winfred Lynn Ward, SR., Fort Myers, FL (US); Ozella Ward, Fort Myers, FL (US)

Correspondence Address: Winfred Lynn Ward Sr., 1828 S. Palmdale Court, Fort Myers, FL, 33916, US

(21) Appl. No.: 12/319,778

(22) Filed: Jan. 13, 2009

(51) Int. Cl. A61L 9/04; B67D 5/08

(52) U.S. Cl. 239/51.5; 239/71

(57) ABSTRACT

The invention pertains to a fragrance distribution system. The system includes a fragrance control chamber including a removable fragrance cartridge having a fluid fragrance located therein. The cartridge has located therein a drum having a wick wound thereon. A dial outside of the cartridge controls the extent of the wick standing in an upward direction within the chamber and being subjected to a flow of air being generated by way of a fan located in the chamber. The speed of the fan is controlled by an adjusting dial located on the exterior of the chamber. There is further an air volume control dial located on the exterior of the chamber which controls the volume of the air being expelled from the chamber into a duct system of an air conditioning system.

### BACKGROUND OF THE INVENTION

The invention pertains to the field of a central scent distribution system that delivers fragrances throughout the house and the building from a central location. The central delivery of the scent is uniquely designed with a wide variety of fragrance ranges from conventional aromas to therapeutic oils. The invention further pertains specifically to dispensing selected types of and quantities of scents from fragrances into an existing heating-ventilation or air conditioning system employing duct works that can be used for the distribution of such fragrances.

Occupants of buildings are concerned with the quality of the ambient indoor air and several devices or systems are deployed or have been developed for masking offense odors to thereby improve the indoor quality of the air. Some devices are hand-held and must be manually operated. In other indoor situations, it is simply desirable to improve the surrounding ambient air in a building by automatically dispensing fragrances at selected times or selected quantities throughout the building from a central location.

U.S. Pat. No. 5,030,253 discloses a fragrance air supplying system. The fragrance is supplied through mist generating means either by blowing air or by vibration produced oscillating means. The mist is blown to a surface portion formed at the end of the fragrant-air separating means which separates evaporated fragrant air from the mist.

U.S. Pat. No. 3,633,881 illustrates a self contained system where a container encloses a fragrance and a centrifugal fan delivers a deodorant into a duct work by way of a wick that is submerged in the container. The system includes a timer that will control the operation of the system at predetermined time intervals.

U.S. Pat. No. 5,924,597 teaches a fragrance dispensing apparatus for use in a multi-room building having an existing HVAC system ventilated by a forcing fan. The apparatus includes a plurality of fragrance containers, a multiple of solenoids, a plurality of programmable timers and a single fan timer. Each fragrance container is controlled by a separate solenoid which in turn is controlled by a separate timer.

### BRIEF DESCRIPTION OF THE DRAWINGS

FIG. 1 illustrates the outside of a fragrance control chamber;

FIG. 2 shows the inside of the control chamber of FIG. 1 including the various control elements;

FIG. 3 shows the control chamber in combination with air ducts.

### DETAILED DESCRIPTION OF THE INVENTION

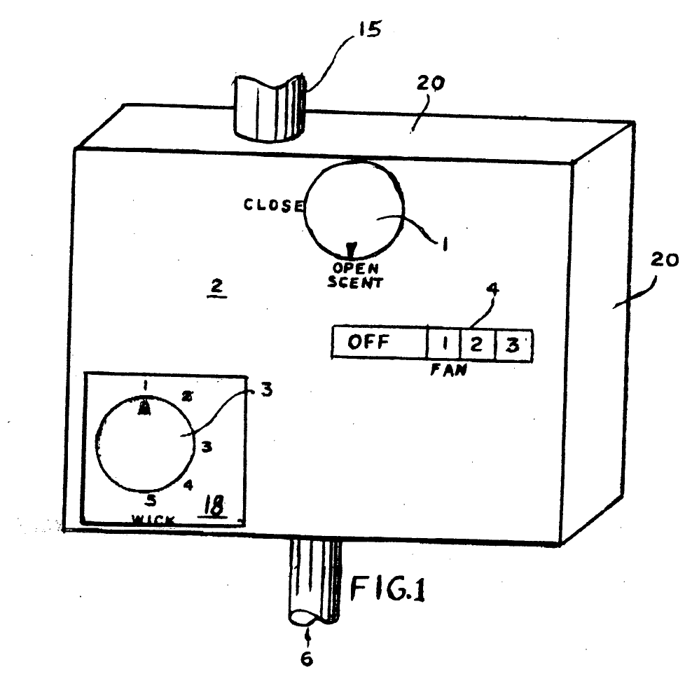

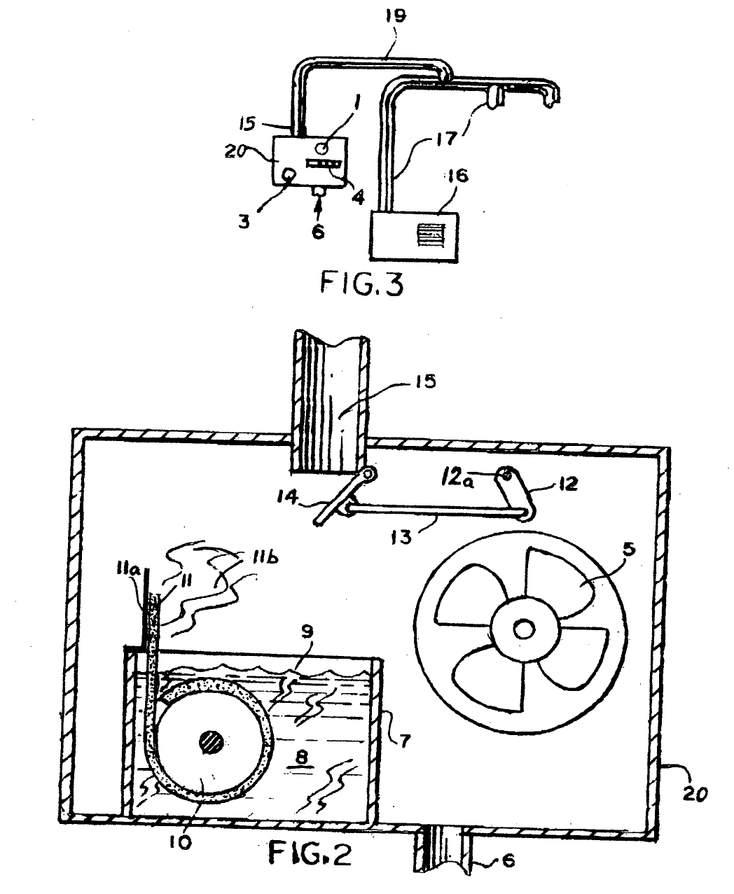

FIG. 1 shows the exterior of a 20 chamber that encloses all the control elements that are involved in the central scent distribution system. In FIG. 1, the exterior surface of the control chamber 20 is shown at 2 which has a dial 3 on an exterior surface of a removable cartridge 18. This dial 3 controls a wick drum on the interior of the cartridge 18 which will be described by having reference to FIG. 2. As can be seen the dial 3 has various gradient numbers thereon which indicate the status of the wick and the amount of the wick being exposed to an air flow. The exterior surface 20 has another dial 1 thereon which will control the amount of air flow that will be advanced into the duct system shown in FIG. 3. The dial 1 shows an open state and a closed state. Of course, any position there between indicates the amount of air flowing into the system. Then there is a third dial or indicator 4 on the exterior surface 2 of the chamber 20 which will indicate by way of gradient numbers the speed of the fan and thereby indicate the volume air flowing into the system. At the bottom of the chamber 20 there is an inlet 6 which will admit the ambient air into the chamber 20 and on top of the chamber 20 there is an outlet pipe 15 which will admit air into the duct system of the air conditioning system.

FIG. 2 illustrates the interior of the chamber 20 and the function of the various control elements. At the bottom of the control chamber 20 there is located a fragrance container 7 in the form of the cartridge 18, (FIG. 1), having the fragrance 8 contained therein in a fluid form. The level of the fragrance fluid 8 in the cartridge 18 is shown at 9. Within the fluid 8 there is located a drum 10 which has a certain supply of a wick material wound thereon. The wick itself should be made of a somewhat stiff material so that when the wick 11 is unwound from the drum it will stand up when it exits above the fluid level 9 and exposes itself to an air fluid flow shown at 11b. In addition, the wick 11 is supported by an upstanding wall 11a which is made of a thin wire mesh material. The purpose of this wall 11a is to aid the wick 11 in its upright position. This way, the wick 11 cannot be blown over by the force of the air coming from the fan 5. The wick 11 will merely lean against the wall 11a while a fragrance scent vapor is released there from. On the other hand, the wick should be porous enough to easily pick up the air coming from the fan 5 and release any scent evaporations there from. It is preferred to make this wick material in a somewhat flat construction to increase its area of exposure to the air flow. The wick could also be made of a braided material because this structure would also increase the fluid flow because a braided material, by nature, is somewhat porous but at the time could also be resistant to bending. The material itself should be a cotton or a rayon known for their infinity to fluid material. As described with reference to FIG. 1, the drum 10 is controlled by the dial 3 located on the front surface of the cartridge 18.

Also contained in the control chamber is a fan 5 which could be constructed as a centrifugal fan or as a vane fan as long as a sufficient air flow is created. The speed of the fan is controlled by the gradient numbers of the slide 4 on front 2 of the chamber 20.

Also shown on the interior of the chamber 20 is an exit control system that will control the amount of air that is driven into the duct system 19 of the air conditioning system shown FIG. 3. To this end, the valve control is controlled by the dial 1 on the exterior surface 2 of the control chamber 20. The dial 1 has a shaft 12a penetrating through the wall of the surface 2 to which is connected a lever 12 which has connected at end thereof a rod 13 which in turn is connected to a flapper valve 14. The flapper valve 14 has the same contour as the diameter of the air exit pipe 15. By turning the dial 1 on front of the chamber 20, the flapper valve 14 more or less will approach the bottom of the air outlet pipe 15 to thereby allow more or less fragrance laden air to enter the distribution system shown in FIG. 3. Of course, any other type of linkage system between the dial 1 and the flapper valve 14 may be employed resulting in the same operational values.

FIG. 3 illustrates one example how the fragrance control chamber may be incorporated into an air conditioning system. In FIG. 3 the fragrance control chamber 20 is connected via the air exit pipe 15 and the fan 5 forces the air past the exposed wick 11, while picking up the fragrance, into the air exhaust pipe 15 and from there into the duct 19 of the air conditioning system and from there into the individual pipes or ducts 17 which lead to the various rooms having air outlets 16.

The fragrance control chamber may be located in various specialized or convenient places. It is suggested that the fragrance control chamber 20 be mounted or placed within a wall with only the front surface 2 showing and with only the front control dials being seen and ready for use by a user. This location could be close to where the thermostat of the air conditioning system is located. It is also suggested that the air passing into the chamber 20 through the inlet pipe 6 be first passed through an air filter.

It should also be noted that the cartridge 18 shown in FIG. 1 is removably mounted within the fragrance control chamber 20. This assures that the level of the fluid fragrance 8 in the cartridge can be observed and can be replenished if necessary.

What we claim is:

1. A fragrance distribution system comprising: a control chamber in the form of an enclosure having a front surface, said front surface having a wick indicator dial on a surface of a removable cartridge located in said control chamber thereon, said front surface further having an air volume adjusting dial thereon and further having an air fan speed indicator dial thereon.

2. The distribution system of claim 1, wherein said wick indicator dial is connected to an interior drum located in said cartridge having a wick contained thereon, said wick is being extended upwardly when said wick indicator dial is turned in a predetermined direction.

3. The distribution system of claim 2, wherein said drum having said wick thereon is located in said cartridge having a fluid fragrance therein.

4. The distribution system of claim 3, wherein said wick has a flat and stiff construction.

5. The distribution system of claim 4 including a mesh wall upstanding from said container to aid said wick to maintain its upright position when air is blown against said wick.

6. The distribution system of claim 1, wherein said air volume adjusting dial is connected to an interior flapper valve mechanism controlling the flow of air out of said enclosure.

7. The distribution system of claim 1, wherein said air speed indicator dial is connected to an interior rheostat controlling the speed of an air fan located inside said enclosure.

8. The distribution system of claim 1, wherein said enclosure has an air inlet and an air outlet.

9. The distribution system of claim 8, wherein said air outlet of said enclosure is connected to a duct system of an air conditioning system.
